(19) United States
(12) Patent Application Publication
(10) Pub. No.: US 2010/0081851 A1
(43) Pub. Date: Apr. 1, 2010

(54) ADSORBENT FOR DRYING ETHANOL

(75) Inventors: Alan P. Cohen, Palatine, IL (US); Thomas M. Reynolds, Mobile, AL (US); Mark M. Davis, Highland Park, IL (US)

Correspondence Address: HONEYWELL/UOP;PATENT SERVICES, 101 COLUMBIA DRIVE, P O BOX 2245 MAIL STOP AB/2B, MORRISTOWN, NJ, 07962, US

(73) Assignee: UOP LLC, Morristown, NJ (US)

(21) Appl. No.: 12/556,877

(22) Filed: Sep. 10, 2009

Related U.S. Application Data
(60) Provisional application No. 61/101,217, filed on Sep. 30, 2008.

(51) Int. Cl. C07C 29/76; G01N 5/02

(52) U.S. Cl. 568/917; 73/73

(57) ABSTRACT

This invention involves the use of a more effective adsorbent to dehydrate ethanol. The most common use for the ethanol is an additive to gasoline. The preferred adsorbent is a type 3A adsorbent that has been ion exchanged with potassium at a level of about 0.6. Surprisingly, this adsorbent has a significantly improved resistance to damage by water upset events.

### CROSS-REFERENCE TO RELATED APPLICATION

This application claims priority from Provisional Application No. 61/101,217 filed Sep. 30, 2008, the contents of which are hereby incorporated by reference.

### BACKGROUND OF THE INVENTION

The present invention is directed to the purification of alcohol and, more particularly, to a process, an adsorbent and a process for identification of an adsorbent for the removal of water from mixtures of alcohol and water and the resulting production of dry alcohol. In certain cases, dry alcohol can be used as a fuel additive or even as the primary component in fuel, to reduce reliance on fossil fuels.

Fuel grade ethanol is being produced from bio-feedstocks such as corn, wheat, sugar cane, sugar beets and barley to augment the hydrocarbon fuels used in automobiles. Ethanol Producer Magazine reports that production of ethanol in the U.S. increased from 18.5 billion liters (4.9 billion gallons) in 2006 to 24.6 billion liters (6.5 billion gallons) in 2007. It further reports that the number of ethanol production plants in the U.S. grew from 117 in 2006 to 159 in 2007. In addition, 48 ethanol plants were under construction in the U.S. in the spring of 2008.

Such increasing production is driven by the high price of gasoline, U.S. government subsidies for production of fuel ethanol, and the U.S. Renewable Fuel Standard program, which, under current regulations, will increase the volume of renewable fuel required to be blended into gasoline to 136 billion liters (36 billion gallons) by 2022.

In the production of fuel grade ethanol from corn, the corn kernels are treated by milling, etc, to separate the starch. Similar steps are taken to treat other bio-feedstocks. Using enzymes, the starch is converted to sugar. The sugar is then fermented to produce ethanol. The ethanol is separated and concentrated to an ethanol-water mixture in the “beer still.” The ethanol water mixture (approximately 40 to 60 wt-% water) in the overhead of the beer still is then concentrated to near the azeotropic concentration of 4 vol-% water in ethanol (192 proof) by distillation in the rectifier column. In practice, the mixture from the rectifier is about 8 vol-% water because concentrating to the azeotropic concentration consumes excessive amounts of energy.

The overhead vapor of the rectifier column is superheated and fed to the dehydrator. The dehydrator dries the ethanol to meet the water specification for fuel grade ethanol of 1 vol-% maximum. Typically, the dehydrator is operated to produce about 5000 to 7000 ppm by volume of water in ethanol to remain well within the specification but to still allow for water absorption in shipping and handling. It is necessary to dehydrate the ethanol to prevent separation of the ethanol from the gasoline in typical blends of up to 10% ethanol in gasoline.

The dehydrator is typically a two-bed vacuum-pressure swing adsorption process using type 3A molecular sieve. Both high and low pressure systems have been employed. In both cases the desorption pressure is below atmospheric pressure. In high pressure systems, ethanol-water vapor from the rectifier column at about 3.8 bar absolute (55 psia) is superheated to about 135° C. The gas flows through an adsorbent bed where water is adsorbed. Concentrated ethanol superheated vapor flows out the effluent end of the bed. The ethanol product is condensed and sent to product storage. A portion of the product effluent is used to purge the bed on regeneration at a pressure of about 0.14 bar absolute (2 psia). At the reduced pressure water desorbs from the adsorbent and is carried out of the bed to a condenser and a vacuum device, such as a vacuum pump or steam ejector. The condensed regeneration effluent is pumped back to the rectifier column.

The process of a lower pressure system is substantially the same, except the feed ethanol-water vapor from the rectifier is at about 1.4 bar absolute (20 psia). The vapor is superheated to about 105° C. In the lower pressure system, the desorption pressure is typically about 0.07 to 0.14 bar absolute (1 to 2 psia).

In a typical 2-bed ethanol drying VPSA (vacuum pressure swing adsorption) cycle, adsorption step time is about 6 minutes. Desorption step time is about 4 minutes. Pressurization and depressurization step times are each about 1 minute. Thus, while bed A is on the adsorption step, bed B undergoes depressurization to the regeneration pressure for about 1 minute, followed by purging with ethanol product from bed A for about 4 minutes, followed by repressurization to the feed pressure with product gas for about 1 minute. Then the flows are switched to place bed B in the adsorption step, and bed A proceeds with depressurization, purging, and repressurization. The cycle continues in this manner with flows switching back to bed A on adsorption, and so on.

The dehydrator may also be a three-bed vacuum-pressure swing adsorption process using type 3A molecular sieve. In a 3-bed ethanol drying VPSA cycle, the process is substantially the same, but the additional bed allows more time for the desorption and pressure change steps while one bed is on adsorption.

As in any process plant, it is desired to reduce the overall investment cost and operating cost needed to produce a given flow of dehydrated on-spec ethanol. The investment cost of the dehydrator unit of the ethanol plant can be reduced by reducing the size of the dehydrator adsorbent beds. A reduction in bed size yields savings in the size of the adsorber vessels needed and in the amount of adsorbent inventory required to fill the beds.

A reduction in adsorber bed size further results in reduced operating costs. Smaller beds reduce the pressure drop of both the adsorption flow and regeneration (or purge) flow. Since the regeneration flow is driven in part by a vacuum device, lower pressure drop results in reduced power consumption by the vacuum device. A lower pressure drop on regeneration also results in a lower regeneration pressure, which results in a higher purge factor (purge factor is volumetric purge/feed ratio) and a more efficient separation.

Bed size reduction also leads to higher recovery of ethanol in the product since the void volume of the bed is lower and, therefore, less ethanol is lost to the desorption effluent on each cycle. Since the desorption effluent is recycled to the distillation section, higher ethanol recovery leads to lower recycle and lower energy consumption.

A reduction in adsorber bed size can be achieved by improving the properties of the adsorbent used in the dehydrators and by optimizing the bed design.

UOP manufactures molecular sieve grade 3A-AG 3 mm (⅛ inch) pellets for use in these VPSA dehydrators. Zeochem manufactures grade Z3-03 4×8 beads for this application. Both of these products are type 3A zeolite potassium sodium aluminosilicates with general composition xK₂O.(1−x)Na₂O.Al₂O₃.2SiO₂.4.5H₂O combined with clay-type binding materials, formed into cylindrical or spherical shapes, and calcined to harden the binder and activate the zeolite.

Zeolite molecular sieves have a crystalline structure that is well understood. The crystals have micropores with dimensions on a molecular scale leading to cavities with adsorption surfaces. Thus, type 4A zeolite with composition Na₂O.Al₂O₃.2SiO₂.4.5H₂O has micropores with effective diameter of approximately 4 Angstroms (0.4 nanometers). Type 3A zeolite with composition xK₂O.(1−x)Na₂O.Al₂O₃.2SiO₂.4.5H₂O has micropores with effective diameter of approximately 3 Angstroms (0.3 nanometers). The parameter “x” which can take a value from zero to unity is the ion exchange ratio or ion exchange level. Such synthetic zeolites with micropores on the nanometer scale adsorb molecules with diameters smaller than the effective micropore diameter but do not adsorb (i.e., they exclude) molecules with diameter larger than the micropore effective diameter.

Although various commercial type 3A zeolite molecular sieve products have similar compositions, they can differ in the effective diameter of the pore opening as a result of differences in their process of manufacture. It is known to those skilled in the art that the level of ion exchange of potassium for sodium, binder selection, heat and steam treatment, and chemical post treatment, as well as other variables, can be used to produce a type 3A molecular sieve adsorbent with an effective pore opening diameter within a more or less narrow range of values.

This important property, known as the molecular sieving effect, is put to good use in the separation of components of numerous fluids. In particular it is used to dry, i.e., to adsorb water from, air, natural gas, ethylene, fluorocarbon refrigerants, petrochemicals, and other fluids. The type 3A micropore will admit water molecules and exclude many other molecules. In so doing the larger molecules are prevented from coadsorbing, that is, competing with water for adsorption on the available sites within the zeolite crystal cavities. If the larger molecules have a strong affinity for zeolite adsorption sites, as is the case with many polar molecules, excluding them produces a major advantage. The advantage is greater equilibrium water loading of the adsorbent especially when drying fluids to lower levels of water. In drying ethanol, excluding the ethanol molecule reduces the coadsorption effect and increases the equilibrium water loading of the zeolite adsorbent.

The effective pore opening diameter influences not only the molecular sieving effect and coadsorption but also dynamic adsorption processes. In particular it influences mass transfer rates by limiting the rate of diffusion of molecules into and out of the zeolite cavity through the pore. In general the smaller the pore, the lower the rate of diffusion, and as the pore opening approaches the effective diameter of the molecule, the diffusion limitation may become very severe. If the pore is made too small, the equilibrium water loading advantage achieved by limiting coadsorption may be partially or completely negated by reduced rates of mass transfer when the zeolite is used in a commercial or test unit. However, even when an effective adsorbent is identified we have found that such adsorbents may be significantly damaged by upset conditions during exposure to liquids. A method for use and selection of an effective adsorbent that will be used under operating conditions that include upset situations has now been discovered.

### SUMMARY OF THE INVENTION

The present invention involves a process for separating ethanol from a feed mixture comprising ethanol and water. The process comprises contacting, at adsorption conditions, the mixture with a type 3A adsorbent that has been ion exchanged at a level of greater than about 0.5, selectively adsorbing the water to the substantial exclusion of ethanol, and thereafter recovering high purity ethanol. In addition to using an adsorbent that is appropriately selective for the given application, applicants have found that it is important to consider the effect of upset conditions that may occur during operations. Such conditions involve exposure of the adsorbent beds to liquid conditions instead of the normal vapor phase conditions. Exposure to liquids has been found to have a deleterious effect upon the capacity of an adsorbent to adsorb one substance in preference to a second substance even after regeneration of the adsorbent bed. Water-containing streams, especially at elevated temperatures can have this effect. In the drying of an ethanol stream containing a high percentage of water, the opportunity for water upset conditions exist. Surprisingly, certain adsorbents have been found to provide an unexpected level of protection against water upset conditions. The prior art zeolites that are exchanged at about the 0.4 level initially will perform at an acceptable level in dehydrating ethanol. However, after exposure to liquid upset conditions, such adsorbents have a significantly lowered capacity for removal of water from ethanol. The preferred type 3A zeolite adsorbents that are used in the present invention have been exchanged with potassium ions at a level from about 0.5 to 0.99. More preferably, the exchange level is about 0.6. The type 3A zeolite adsorbent has a water adsorption capacity greater than about 15% and more preferably greater than about 18%. The type 3A zeolite adsorbent has an ethanol adsorption capacity measured at 121° C. (250° F.) at the vapor pressure of ethanol at 0° C. of less than about 4% and more preferably less than about 2%.

Another aspect of the present invention involves a process to identify a useful adsorbent for purification of a feed stream comprising a) contacting an adsorbent with said feed stream wherein said adsorbent adsorbs a determined amount of one component of said feed stream while adsorbing a second determined amount of a second component; b) then contacting said adsorbent with an excess amount of a liquid; c) then drying said liquid from said adsorbent and then repeating step a); d) then comparing the determined amounts from steps a) and c); e) repeating steps a) through d) with at least two different adsorbents; f) comparing the determined amounts of said one component and determined measured amount of said second component for each of said at least two different adsorbents and then selecting said useful adsorbent based upon a determination of which of the at least two different adsorbents continued to adsorb a maximum measured amount of said one component while said second measured amount of said second component is at a minimum level.

### DETAILED DESCRIPTION OF THE INVENTION

The present invention involves a process for separating ethanol from a feed mixture comprising ethanol and water. The process comprises contacting, at adsorption conditions, the mixture with a type 3A adsorbent that has been ion exchanged at a level of greater than about 0.5, selectively adsorbing the water to the substantial exclusion of ethanol, and thereafter recovering high purity ethanol. In addition to using an adsorbent that is appropriately selective for the given application, applicants have found that it is important to consider the effect of upset conditions that may occur during operations. Such conditions involve exposure of the adsorbent beds to liquid conditions instead of the normal vapor phase conditions. Exposure to liquids has been found to have a deleterious effect upon the capacity of an adsorbent to adsorb one substance in preference to a second substance even after regeneration of the adsorbent bed. Water-containing streams, especially at elevated temperatures can have this effect. In the drying of an ethanol stream containing a high percentage of water, the opportunity for water upset conditions exist. Surprisingly, certain adsorbents have been found to provide an unexpected level of protection against water upset conditions. The prior art zeolites that are exchanged at about the 0.4 level initially will perform at an acceptable level in dehydrating ethanol. However, after exposure to liquid upset conditions, such adsorbents have a significantly lowered capacity for removal of water from ethanol. The preferred type 3A zeolite adsorbents that are used in the present invention have been exchanged with potassium ions at a level from about 0.5 to 0.99. More preferably, the exchange level is about 0.6. The type 3A zeolite adsorbent has a water adsorption capacity greater than about 15% and more preferably greater than about 18%. The type 3A zeolite adsorbent has an ethanol adsorption capacity measured at 121° C. (250° F.) at the vapor pressure of ethanol at 0° C. of less than about 4% and more preferably less than about 2%.

### Example 1

Samples of type 3A zeolite molecular sieve agglomerates were tested in a 2-bed vacuum-pressure swing adsorption ethanol drying pilot plant. The adsorbent beds were 51 mm (2 inches) in internal diameter and 1.22 meters (48 inches) tall, mounted vertically. An average of 1933 grams of molecular sieve were loaded into each bed, varying slightly depending on the bulk density of the samples. A mixture of 91.2 wt-% ethanol and 8.8 wt-% water was vaporized and superheated and fed to the adsorption unit at a pressure of 3.8 bar absolute (55 psia) and temperature of 135° C.

The unit was operated with a fixed purge flow of approximately 1.5 grams/minute for 150 seconds after evacuation for 50 seconds. The feed flow was then adjusted to make a product effluent with approximately 4000 ppm/wt water in ethanol. The average feed flow was 23.0 grams/minute and the adsorption step time was 4.5 minutes.

The productivity under these conditions was calculated as the feed flow (in grams/hour) divided by the weight of one adsorbent bed (in grams). Subtracting the mass of water exiting the adsorbent bed from the mass of water entering the adsorbent bed in one adsorption step gives the amount of water (grams/cycle) adsorbed during the adsorption step. Dividing by the weight of one bed (grams) gives the differential loading.

Under a fixed set of conditions, the differential loading conveys the same information as the productivity. Higher values of either parameter are beneficial. The higher the value of either productivity or differential loading, the lower the inventory of adsorbent required to meet the dryness specifications under the given conditions. The lower the inventory of adsorbent, the smaller are the vessels required, and the greater the benefits described above. Recovery is the mass of ethanol produced divided by the amount of ethanol fed to the VPSA system. Drying performance is thus measured in an ethanol drying pilot plant at a consistent set of operating conditions and is expressed in terms of differential loading (grams of water adsorbed per 100 grams of adsorbent) and/or productivity (grams of dried ethanol product made per hour per gram of adsorbent) and/or bed size factor (grams of adsorbent required per gram/minute of feed).

### Example 2

Various commercial and laboratory prepared samples of zeolite type 3A were tested according to the method in Example 1. Differential loadings of 2.2 to 3.4 g/100 g were observed under the test conditions. In these experiments the product recovery averaged 89%.

In some cases the targeted product effluent of 4000 ppm/wt water in ethanol was not achieved in the experimental work. In these cases the parameters of productivity and differential loading were obtained by careful interpolation or extrapolation of the data. [End of example.]

Ethanol drying adsorbents are designed with pores small enough to (at least partially) exclude ethanol molecules yet large enough to admit water molecules to the internal adsorption sites of the zeolite molecular sieve crystals. Such exclusion reduces ethanol coadsorption which reduces competition of ethanol for the internal adsorption sites. In other words, exclusion provides higher selectivity of the adsorbent for water over ethanol, which in turn optimizes water capacity and drying performance.

The feed to VPSA (vacuum pressure swing adsorption) ethanol dehydrator adsorber beds is a mixture of superheated water and ethanol vapors. Upset conditions occasionally occur where water-ethanol liquid mixtures flow to the dehydrator beds instead of vapors. The high water content of the liquid overloads and suddenly saturates the adsorbent. Moreover, the desorption steps of the VPSA process cycle are rendered inoperable, resulting in a failure of the dehydrator to produce dry ethanol.

Following such an upset, the adsorber beds are eventually returned to normal vapor phase operation. The beds may be purged with hot dry ethanol to recover their drying performance or more simply drained of liquid and returned to their normal operation without special purging. It has been observed that sometimes the dehydrator adsorber beds do not fully recover their prior drying performance after returning to normal operation, even after many days of operation and many, many VPSA cycles.

The consequence of the described upset condition has been simulated in the laboratory by immersing activated adsorbent in liquid water for one hour followed by drying the adsorbent by heating in an oven for 18 hours at 115° C. and then reactivating the adsorbent by further heating the adsorbent in an oven for one hour at 575° C. The hot ethanol adsorption (at 121° C. [250° F.] and the vapor pressure of pure ethanol at 0° C. by the McBain method) is measured before and after this immersion-drying-reactivation procedure.

A more stable adsorbent will have a smaller (or zero) increase in hot ethanol adsorption upon subjecting it to the immersion-drying-reactivation procedure. Conversely, a less stable adsorbent will have a larger increase in hot ethanol adsorption upon subjecting it to the immersion-drying-reactivation procedure.

If the upset condition causes the molecular sieve pores to open and co-adsorption of ethanol to increase, then inferior drying performance results as ethanol competes with water for the available adsorption sites. Furthermore, the inferior performance persists and the dehydrator adsorber beds do not fully recover their original drying performance.

### Example 3

In the TABLE, the hot ethanol adsorption data (at 121° C. [250° F.]) and the vapor pressure of pure ethanol at 0° C. by the McBain method) is shown in rows B and C with the potassium exchange (row A) of the zeolite material used in the beads. Row B (Ethanol adsorption before immersion) is the ethanol adsorption of fresh molecular sieve beads. Row C (Ethanol adsorption after immersion and reactivation) is the ethanol adsorption of molecular sieve beads subjected to one cycle of the immersion-drying-reactivation procedure. The increase is indicated in row D.

The TABLE illustrates that high potassium exchange (0.6 for Samples 1 and 2) produces a more stable type 3A adsorbent bead as it protects type 3A adsorbents from pore opening when subjected to upset conditions. In contrast the lower potassium exchange (0.26 and 0.35 for Samples 1 through 4) produces less stable adsorbent beads. The upset condition is simulated as a sudden re-hydration by immersion of activated beads in liquid water followed by drying and reactivation of the adsorbent.

Surprisingly, adsorbent materials, all with low initial ethanol adsorption, differ in stability due to prior treatment by potassium exchange.

The observed pore size stability of the molecular sieve is thus valuable in protecting VPSA ethanol plant dehydrator adsorbents from lasting damage from process upsets. Type 3A dehydrator adsorbents made with high potassium exchange recover from process upsets with substantially all of their previous water capacity and drying performance.

### Example 4

It is useful to define the exclusion of ethanol as: Water adsorption capacity minus ethanol adsorption capacity where water adsorption capacity is given by the McBain method at room temperature and vapor pressure of water at 0° C. and the ethanol adsorption capacity as given in Example 3. The ethanol exclusion data are given in the TABLE and the retained ethanol exclusion is given in row G. The data show that samples with high potassium exchange have nearly complete retention of the ethanol exclusion property.

TABLE
Adsorption and Exclusion of Ethanol from Type 3A Molecular Sieves
Before and After Immersion in Liquid Water*

| | | Sample | | | | | |
|---|---|---|---|---|---|---|---|
| | | 1 | 2 | 3 | 4 | 5 | 6 |
| A | Potassium exchange, | 0.26 | 0.26 | 0.35 | 0.35 | 0.6 | 0.6 |
| | K/(K + Na) | | | | | | |
| B | Ethanol adsorption | 0.6 | 1.5 | 1.2 | 2.1 | 0.3 | 0.4 |
| | before immersion, wt % | | | | | | |
| C | Ethanol adsorption | 9.1 | 9.4 | 6.2 | 5.4 | 0.5 | 0.5 |
| | after immersion, wt-% | | | | | | |
| D | Increase in ethanol | 8.5 | 7.9 | 5.0 | 3.3 | 0.2 | 0.1 |
| | adsorption, wt-% | | | | | | |
| E | Ethanol exclusion | 17.6 | 16.7 | 16.5 | 16.7 | 18.5 | 16.4 |
| | before immersion, wt-% | | | | | | |
| F | Ethanol exclusion | 9.1 | 8.8 | 11.5 | 13.4 | 18.3 | 16.3 |
| | after immersion, wt-% | | | | | | |
| G | Ethanol exclusion | 52% | 53% | 70% | 80% | 99% | 99% |
| | retained | | | | | | |

*According to the procedure of Example 4

### Example 5

The performance in terms of differential loading of samples tested in the pilot plant described in Examples 1 and 2 varied with the degree of ethanol adsorption. A sample with hot ethanol adsorption of 0.7% performed with a differential loading of 3.4 grams of water per hundred grams of adsorbent, while a sample with hot ethanol adsorption of 3.0% performed with a differential loading of 2.4 grams of water per 100 grams of adsorbent. The two results, both taken under the high operating pressure conditions, show that even a moderately higher ethanol adsorption results in a 30% loss in performance. The results emphasize the importance of maintaining the ethanol exclusion property after process upset conditions.

1. A process for drying ethanol comprising contacting said ethanol with a type 3A zeolite adsorbent that has been exchanged with potassium ions at a level between 0.5 to 0.99.

2. The process of claim 1 wherein said type 3A zeolite adsorbent has been exchanged with potassium ions at a level of about 0.6.

3. The process of claim 1 wherein said type 3A zeolite adsorbent maintains at least 80% of its capacity for adsorption of water upon saturation level exposure to water containing liquids.

4. The process of claim 1 wherein said type 3A zeolite adsorbent maintains at least 85% of its capacity to exclude ethanol from adsorption after saturation level exposure of said type 3A adsorbent to water-containing liquids.

5. The process of claim 1 wherein said type 3A zeolite adsorbent has a water adsorption capacity greater than about 15%.

6. The process of claim 1 wherein said type 3A zeolite adsorbent has a water adsorption capacity greater than about 18%.

7. A process to identify a useful adsorbent for purification of a feed stream comprising:
a) contacting an adsorbent with said feed stream wherein said adsorbent adsorbs a determined amount of one component of said feed stream while adsorbing a second determined amount of a second component;
b) then contacting said adsorbent with an excess amount of a liquid;
c) then drying said liquid from said adsorbent and then repeating step a);
d) then comparing measurements from steps a) and c);
e) repeating steps a) through d) with at least two different adsorbents; and
f) comparing the determined amounts of said one component and determined measured amount of said second component for each of said at least two different adsorbents and then selecting said useful adsorbent based upon a determination of which of the at least two different adsorbents continued to adsorb a maximum measured amount of said one component while said second measured amount of said second component is at a minimum level.

8. The process of claim 7 wherein said liquid is water.

9. The process of claim 7 wherein said adsorbent is a type 3A adsorbent.

10. The process of claim 9 wherein said adsorbent is exchanged with potassium at a level from about 0.5 to 0.99.

11. The process of claim 10 wherein said adsorbent is exchanged with potassium at a level of about 0.6.
